(19) United States
(12) Patent Application Publication
(10) Pub. No.: US 2003/0167579 A1
(43) Pub. Date: Sep. 11, 2003

(54) Oxidation dyeing composition for keratinous fibres and dyeing method using same

(76) Inventor: Gerard Lang, Saint Prix (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/333,664

(22) PCT Filed: Mar. 5, 2001
(86) PCT No.: PCT/FR01/00646

(30) Foreign Application Priority Data
Mar. 6, 2000 (FR) 00/02860

(51) Int. Cl. A61K 7/13

(52) U.S. Cl. 8/405; 8/406; 8/408; 8/415; 8/421

(57) ABSTRACT

The invention concerns a ready-to-use oxidation dyeing composition for keratinous fibres, and in particular human keratinous fibres such as hair comprising, in a suitable dyeing medium, at least an oxidation base selected among certain substituted paraphenylenediamine derivatives and their addition salts with an acid, at least an alkaline agent and hydrogen peroxide, and the dyeing method using said composition.

The invention relates to a ready-to-use composition for the oxidation dyeing of keratin fibers, and in particular of human keratin fibers such as the hair, comprising, in a medium that is suitable for dyeing, at least one oxidation base chosen from certain substituted para-phenylenediamine derivatives and the addition salts thereof with an acid, at least one selected alkaline agent and hydrogen peroxide, and also to the dyeing process using this composition.

It is known practice to dye keratin fibers, and in particular human hair, with dye compositions containing oxidation dye precursors, in particular ortho- or para-phenylenediamines, ortho- or para-aminophenols, and heterocyclic bases, which are generally known as oxidation bases. These oxidation dye precursors (oxidation bases) are colorless or weakly colored compounds which, when combined with oxidizing products, can give rise to colored compounds and dyes by a process of oxidative condensation.

It is also known that the shades obtained with these oxidation bases can be varied by combining them with couplers or coloration modifiers, the latter being chosen in particular from aromatic meta-diamines, meta-aminophenols, meta-diphenols and certain heterocyclic compounds.

The variety of molecules used as oxidation bases and couplers allows a wide range of colors to be obtained.

The so-called “permanent” coloration obtained by means of these oxidation dyes should moreover satisfy a certain number of requirements. Thus, it should allow shades to be obtained in the desired intensity and it should have good staying power with respect to external agents (light, bad weather, washing, permanent-waving, perspiration or rubbing).

The dyes should also allow gray hair to be covered and, finally, they should be as unselective as possible, i.e. they should allow only the smallest possible differences in coloration to be obtained along the same keratin fiber, which may indeed be differently sensitized (i.e. damaged) between its end and its root.

It has already been proposed, especially in patent applications JP-11-158 046, JP-11-158 047 and JP-11-158 048 to use compositions for the oxidation dyeing of keratin fibers, containing substituted para-phenylenediamine derivatives as oxidation dye precursors. However, the colorations obtained by using these compositions are not always sufficiently intense, chromatic or resistant to the various attacking factors to which the hair may be subjected.

However, the Applicant has now discovered that it is possible to obtain novel dyes, which are capable of giving chromatic, intense, attractive colorations in varied shades, which are relatively unselective and resistant to the various attacking factors to which the fibers may be subjected, by combining at least one oxidation base chosen from certain para-phenylenediamine derivatives of formula (I) defined below and the addition salts thereof with an acid, at least one selected alkaline agent and hydrogen peroxide.

This discovery forms the basis of the present invention.

Thus, a first subject of the invention is a ready-to-use composition for the oxidation dyeing of keratin fibers, in particular of human keratin fibers such as the hair, characterized in that it comprises, in a medium that is suitable for dyeing:

(A) at least one oxidation base chosen from the substituted para-phenylenediamine derivatives of formula (I) below, and the addition salts thereof with an acid:

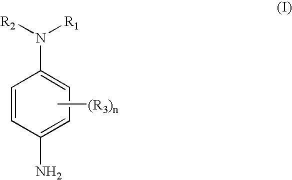

in which:

R₁ and R₂ can take one of the following meanings i) to v) below:

i) R₁ and R₂ simultaneously represent a —(CH₂)₂CHOHCH₂OH radical; or

ii) R₁ represents a —CH₂(CHOH)₄CH₂OH radical and R₂ represents a hydrogen atom, an alkyl or aryl radical or a heterocycle; or

iii) R₁ represents an alkyl or aryl radical or a heterocycle and R₂ represents an alkylene radical —(CH₂)ₘ— in which m is an integer equal to 2 or 3, said alkylene radical forming a ring together with the nitrogen atom, the carbon atom of the benzene ring bearing the nitrogen atom and one of the two carbon atoms of the benzene ring that are adjacent to it, it being understood that when R₁ is an alkyl or aryl radical, then either R₁ or said alkylene radical is substituted with a radical containing at least one nitrogen, oxygen or sulfur atom;

iv) R₁ represents a radical —(CH₂CH₂O)ₚR₄ in which p is an integer between 2 and 8 inclusive, R₄ and R₂, which may be identical or different, represent a hydrogen atom, an alkyl or aryl radical or a heterocycle;

v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;

R₃ represents a halogen atom, an alkyl or aryl radical, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical, a heterocycle or a heterocycle linked to the benzene ring of formula (I) via an ether or thio bond;

said alkyl radicals containing from 1 to 25 carbon atoms and possibly being linear, branched or cyclic and possibly being substituted with one or more radicals, and in this case possibly representing a mono- or polyhydroxyalkyl radical, an alkoxyalkyl radical, an aminoalkyl radical optionally substituted on the nitrogen atom, or a carboxyalkyl, alkylcarboxyalkyl, thioalkyl, alkylthioalkyl, cyanoalkyl, trifluoroalkyl, sulfoalkyl, phosphoalkyl or haloalkyl radical;

said alkoxy radicals containing from 1 to 25 carbon atoms and possibly being linear, branched or cyclic;

said aryl radicals containing from 6 to 26 carbon atoms and possibly being substituted with one or more radicals chosen from alkyl, substituted alkyl and alkoxy radicals;

the heterocycles being monocyclic or polycyclic, each ring being 3-, 4-, 5- or 6-membered and possibly containing one or more hetero atoms, it being understood that in the case of polycyclic heterocycles, at least one of the rings contains at least one hetero atom such as N, O or S;

n is an integer between 0 and 4; it being understood that when n is greater than 1, then the radicals R₃ may be identical or different and may together form a 3-, 4-, 5- or 6-membered saturated or unsaturated ring; with the proviso that:

1) when R₁ and R₂ have the meanings defined in point v), then the compounds of formula (I) do not contain more than 3 hydroxyl radicals;

2) when R₁ and R₂ have the meanings defined in point v) and when R₁ and R₂ form a pyrrolidine ring substituted with a carbamoyl radical on the carbon in the position alpha to the nitrogen atom to which they are attached, then n is other than 0; or the pyrrolidine ring bears at least two substituents;

3) when R₁ and R₂ have the meanings defined in point v) and when R₁ and R₂ form a pyrrolidine ring substituted with a hydroxymethyl radical on the carbon in the alpha position relative to the nitrogen atom to which they are attached and when n=0 or 1, then either said ring bears at least two additional substituents, or said ring bears only one second substituent other than a hydroxyl radical on the carbon located in the β position relative to the nitrogen atom and relative to the carbon bearing said hydroxymethyl substituent; or alternatively, when R₁ and R₂ have the meanings defined in point v) and when R₁ and R₂ form a pyrrolidine ring substituted with a hydroxymethyl radical on the carbon located in the alpha position relative to the nitrogen atom to which they are attached, and when n=1, then R₃ is other than an alkyl or mono- or polyhydroxyalkyl radical;

4) when R₁ and R₂ have the meanings defined in point iii), the compounds of formula (I) must satisfy at least one of the following four conditions:

a) irrespective of the value of n, the alkylene ring formed by the radical R₂ comprises a substituent in addition to the radical R₁; or

b) n is greater than 1; or

c) when n is equal to 1, then R₃ represents an aryl radical or a heterocycle; or

d) when n is equal to 0 or 1, then R₁ represents an aryl radical, a heterocycle or a substituted alkyl radical other than a monohydroxyalkyl radical;

5) R₁ and R₂ form a ring other than piperazine or diazacycloheptane;

(B) at least one alkaline agent chosen from alkanolamines, diaminoalkanes and aqueous ammonia, and

(C) hydrogen peroxide.

The addition salts with an acid of the substituted para-phenylenediamine derivatives of formula (I) that may be used in the dye compositions according to the present invention are chosen especially from the hydrochlorides, hydrobromides, sulfates, tartrates, lactates and acetates.

The ready-to-use dye composition in accordance with the invention gives chromatic, intense, attractive colorations in varied shades, with low selectivity and excellent resistance properties not only with respect to atmospheric agents such as light and bad weather, but also with respect to perspiration and the various treatments to which the hair may be subjected.

For the purposes of the invention, the. expression “ready-to-use composition” means any composition intended to be applied immediately to keratin fibers.

A subject of the invention is also a process for the oxidation dyeing of keratin fibers using said composition.

Another subject of the invention is a multi-compartment dyeing device or “kit”.

A subject of the invention is also a composition for the oxidation dyeing of keratin fibers, and in particular of human keratin fibers such as the hair, comprising, in a medium that is suitable for dyeing, at least one substituted para-phenylenediamine derivative of formula (I) and at least one alkaline agent chosen from alkanolamines, diaminoalkanes and aqueous ammonia.

According to the present invention, the substituted para-phenylenediamine derivatives that are preferably used are those of formula (I) above in which:

R₁ and R₂ can take one of the meanings i) to v) below:

i) R₁ and R₂ simultaneously represent a —(CH₂)₂CHOHCH₂OH radical; or

ii) R₁ represents a —CH₂(CHOH)₄CH₂OH radical and R₂ represents a hydrogen atom or an alkyl radical; or

iv) R₁ represents a radical —(CH₂CH₂O)ₚR₄ in which p is an integer between 2 and 8 inclusive, R₄ and R₂, which may be identical or different, represent a hydrogen atom or an alkyl radical;

v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, which is not in a meta position relative to the nitrogen atom of the heterocycle;

R₃ represents a halogen atom, an alkyl or aryl radical or a heterocycle,

n is an integer equal to 0, 1 or 2.

More particularly, according to the invention, in formula (I), R₁ and R₂ form a pyrrolidine heterocycle.

Among the substituted para-phenylenediamine derivatives of formula (I) above that may be mentioned most particularly are 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis-(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-β-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1-N-(2′,3′,4′,5′,6′-pentahydroxyhexyl)-1-N-(4″-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine, 1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′pentahydroxyhexyl)-3-ethyloxy-para-phenylenediamine, 1-N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)-amino-7amino-1-methylindole and 1-N-(hydroxyethyloxyethyl)-1-N-(2′,3′,4′,5′,6′-pentahydroxyhexyl)-3-ethyl-para-phenylenediamine, and the addition salts thereof with an acid, and among which, the ones that are even more particularly preferred are: 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine and 1-N-(hydroxyethyloxyethyl)-1-N-(2′,3′,4′,5′,6′-pentahydroxyhexyl)-3-ethyl-para-phenylenediamine, and the addition salts thereof with an acid.

Mention may also be made most particularly of 1-N-(3′,4′-dihydroxybutyl)-5-aminoindoline, 1-(2′-hydroxyethyl)-2-methyl-5-aminoindoline, 1-methyl-2-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyloxy-ethyloxyethyloxyethyl-6-isopropyl-5-aminoindoline, 2-hydroxyethyl-3-methyl-5-aminoindoline, 2-hydroxyethyloxyethyloxyethyl)-5-aminoindoline, 1-carboxymethyl-2,3,3-trimethyl-5-aminoindoline, 1-methylsulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, the dimethyl ester 6-amino-1-methyl-1,2,3,4-tetrahydrofuro-[2,3,h]-quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetrahydroquinoline, 6-amino-1-(ethoxyethoxy-ethoxyethoxy-3′,4′-dihydroxybutyl)-2,2,3,7-tetramethyl-1,2,3,4-tetrahydroquinoline, 6-amino-1-(hydroxyethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis-(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyloxyethyloxyethyloxyethyloxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyl)-2,2-dimethyl-4-hydroxymethyl-7isopropyl-1,2,3,4-tetrahydroquinoline, 6-amino-1-(hydroxyethyloxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl) -4-(hydroxyethyloxyethyloxyethyloxy-ethyl)-2,2-dimethyl-7-isopropyl-1,2,3,4-tetrahydroquinoline, 6-amino-1-(hydroxyethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydroquinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline and 6-amino-1-bromo-2,2-dimethyl-4-mercapto-7-isopropyloxy-1,2,3,4-tetrahydroquinoline, and the addition salts thereof with an acid.

Mention may also be made most particularly of 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic acid), 1-(4′-amino-3′-acetylaminophenyl)-3-hydroxymethylpyrrolidine, 7-amino-4-(2′-methyl)-pyrrolidinylbenzofuran, 1-(4′-aminophenyl)-2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropylphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2″′,4″′-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxyethyloxyethyloxyethyloxyethyloxyethyloxy-ethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3-4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxy-ethyloxyethyloxyethyl)-3-isopropyloxy-para-phenylenediamine, 1-N,N-bis(hydroxyethyloxy-ethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(methoxyethyloxyethyloxyethyloxyethyloxyethyloxyethyloxyethyl)-3-methoxy-para-phenylenediamine, 1-N,N-bis(hydroxyethyloxy-ethyloxyethyloxyethyloxyethyloxyethyloxyethyloxyethyl)-3-methyl-para-phenylenediamine, 1,N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine and 1-N,N-bis(benzyloxyethyloxyethyloxyethyl)-3-isopropyl-para-phenylenediamine, and the addition salts thereof with an acid, and among which, the ones that are even more particularly preferred are: 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrollidine, 1-(4′-amino-3′-phenoxyphenyl)-3-methylsulfonamidopyrrolidine, 1-(4′-aminophenyl)-2-(4″-aminophenoxymethyl)piperidine, 1-(4′-aminophenyl)-2-hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxy-ethyloxyethyloxyethyl)-3-methyl-para-phenylenediamine and 1-N,N-bis(benzyloxyethyloxyethyloxyethyl)-3-isopropyl-para-phenylenediamine, and the addition salts thereof with an acid.

The para-phenylenediamine derivative(s) of formula (I) are used as oxidation bases in the composition intended for oxidation dyeing or the ready-to-use oxidation dye composition in accordance with the invention in concentrations ranging from 0.0001% to 20%, preferably from 0.001% to 15% and even more particularly from 0.01% to 10% by weight relative to the total weight of the composition.

For the purposes of the present invention, the term “alkanolamine” means any saturated or unsaturated, linear or branched hydrocarbon compound containing from 2 to 100 carbon atoms and comprising (i) at least one amine function optionally substituted with one or two substituents that are preferably C₁-C₄ alkyl or substituted C₁-C₄ alkyl such as, for example, mono- or polyhydroxyalkyl, and (ii) at least one hydroxyl function not borne by the amine function.

Preferably, the hydrocarbon chain is saturated.

Among the alkanolamines that may be used according to the invention, mention may be made of monoethanolamine, diethanolamine, triethanolamine, triisopropanolamine, 2-amino-2-methyl-1-propanol, 2-amino-2-methyl-1,3-propanediol, 2-amino-2-ethyl-1,3-propanediol, 2-amino-1-butanol, tris(hydroxy-methyl)aminomethane, 2-aminoethylethanolamine, 1-diethylamino-2,3-propanediol, 2-dimethylamino-2-methyl-1-propanol, dimethylethanolamine, diethylethanolamine, ethylmonoethanolamine and methylethanolamine.

Preferably, monoethanolamine is used.

The diaminoalkanes-used according to the invention are preferably those of formula (II) below:

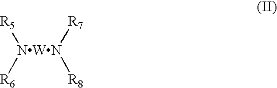

in which W is an alkylene residue optionally substituted with a hydroxyl group or a C₁-C₄ alkyl radical; R₅, R₆, R₇ and R₈, which may be identical or different, represent a hydrogen atom or a C₁-C₄ alkyl or C₁-C₄ hydroxyalkyl radical.

Preferably, W is a propylene residue.

Even more preferentially, the diaminoalkane is diaminopropane.

Among all the alkaline agents that may be used according to the invention, aqueous ammonia is preferably used.

The alkaline agent(s) is (are) present in the ready-to-use oxidation dye composition or the composition intended for oxidation dyeing according to the invention in concentrations ranging from about 0.1% to about 20% by weight and preferably from about 0.5% to about 10% by weight of active material relative to the total weight of the composition intended for oxidation dyeing or the ready-to-use oxidation dye composition.

The pH of the ready-to-use dyeing composition according to the invention is preferably greater than 7 and even more preferentially greater than 8.

The hydrogen peroxide present in the ready-to-use composition according to the invention is used as an oxidizing agent. It is present therein in a weight concentration of between about 0.5% and 15% and preferably between about 1% and 10%, which corresponds to an equivalent concentration of aqueous hydrogen peroxide solution by volume of from about 1.6 to 50 volumes and preferably from about 3.2 to 32 volumes.

The ready-to-use oxidation dye composition or the composition intended for oxidation according to the invention preferably contains at least one coupler.

Among these couplers, mention may be made especially of meta-phenylenediamines, meta-aminophenols, meta-diphenols and heterocyclic couplers such as, for example, indole derivatives, indoline derivatives, benzimidazole derivatives, benzomorpholine derivatives, sesamol derivatives, pyridine, pyrimdine and pyrazole derivatives, and the addition salts thereof with an acid.

These couplers are more particularly chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one and 1-phenyl-3-methylpyrazol-5-one, and the addition salts thereof with an acid.

The coupler(s) may be present in said composition according to the invention in a concentration of between 0.0001% and 15% by weight relative to the total weight of the composition.

The ready-to-use oxidation dye composition or the composition intended for oxidation dyeing according to the invention may also contain at least one additional oxidation base other than the substituted para-phenylenediamine derivatives of formula (I) and/or at least one direct dye.

Among the additional oxidation bases that may be used according to the invention, mention may be made of para-phenylenediamine, para-tolylenediamine, 2-hydroxyethyl-para-phenylenediamine, 1-N,N-bis(2-hydroxyethyl)-para-phenylenediamine, para-aminophenols such as 3-methyl-4-aminophenol and 4-aminophenol, ortho-phenylenediamines, ortho-aminophenols, double bases, heterocyclic bases, for instance pyrimidines such as 2,4,5,6-tetraaminopyrimidine, or pyrazoles, such as 1-(2-hydroxyethyl)-4,5-diaminopyrazole.

The additional oxidation base(s) may be present in a concentration of between 0.0001% and 15% by weight relative to the total weight of said composition.

The medium that is suitable for dyeing (or the support) for the dye composition according to the invention generally consists of water or of a mixture of water and at least one organic solvent to dissolve the compounds that would not be sufficiently water-soluble. Examples of organic solvents that may be mentioned include C₁-C₄ alkanols such as ethanol and isopropanol; glycerol; glycols and glycol ethers, for instance 2-butoxyethanol, propylene glycol, propylene glycol monomethyl ether, diethylene glycol monomethyl ether and monoethyl ether, and also aromatic alcohols, for instance benzyl alcohol or phenoxyethanol, similar products and mixtures thereof.

The solvents may be present in proportions preferably of between 1% and 40% by weight approximately relative to the total weight of the dye composition, and even more preferentially between 5% and 30% by weight approximately.

The dye composition in accordance with the invention may also contain various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners or thickening polymers such as, for example, nonionic guar gums, antioxidants or reducing agents, penetrating agents, sequestering agents, fragrances, buffers, dispersants, conditioners such as, for example, volatile or non-volatile silicones, which are modified or unmodified, film-forming agents, ceramides, preserving agents and opacifiers.

Preferably, the dye composition of the invention contains at least one cationic polymer in a proportion of about from 0.05% to 10% by weight and at least one surfactant, preferably a nonionic surfactant, in a proportion of from 0.1% to 20% by weight. Preferably, it also contains at least one thickening polymer preferably chosen from polymers comprising at least one hydrophilic unit and at least one fatty chain in a proportion of about from 0.05% to 10% by weight.

Said composition may also contain reducing agents or antioxidants. These may be chosen in particular from sodium sulfite, thioglycolic acid, thiolactic acid, sodium bisulfite, dehydroascorbic acid, hydroquinone, 2-methylhydroquinone, tert-butylhydroquinone and homogentisic acid, and they are then generally present in amounts ranging from about 0.05 to 1.5% by weight, relative to the total weight of the composition.

Needless to say, a person skilled in the art will take care to select this or these optional additional compound(s), such that the advantageous properties intrinsically associated with the dye composition in accordance with the invention are not, or are not substantially, adversely affected by the envisaged addition(s).

The ready-to-use dye composition in accordance with the invention may be in various forms, such as in the form of liquids, powders, creams or gels, that may be pressurized, or in any other form that is suitable for dyeing keratin fibers, and especially human hair.

A subject of the invention is also a process for dyeing keratin fibers, and in particular human keratin fibers such as the hair, using the ready-to-use dye composition defined above.

According to this process, a dye composition containing, in a medium that is suitable for dyeing, at least one substituted para-phenylenediamine derivative of formula (I) is mixed, at the time of use, with an oxidizing composition containing, in a medium that is suitable for dyeing, at least hydrogen peroxide present in an amount that is sufficient to develop a coloration, the alkaline agent(s) defined above being present in one or both of the compositions. The mixture obtained is then applied to the keratin fibers and is left in place for 3 to 50 minutes approximately and preferably 5 to 30 minutes approximately, after which the fibers are rinsed, washed with shampoo, rinsed again and dried.

The dye composition before mixing with the hydrogen peroxide may also be in various forms, such as in the form of liquid, cream or gel, or in any other form that is suitable, after mixing, for dyeing keratin fibers, and especially human hair.

Another subject of the invention is a multi-compartment dyeing device or “kit” or any other multi-compartment packaging system, at least one compartment of which contains a dye composition containing at least one substituted para-phenylenediamine derivative of formula (I) defined above and another compartment of which contains an oxidizing composition containing hydrogen peroxide, the alkaline agent(s) defined above being present in one or both of the compositions. These devices may be equipped with a means for applying the desired mixture to the hair, such as the devices described in patent FR-2 586 913 in the name of the Applicant.

The example that follows is intended to illustrate the invention.

### EXAMPLE 1

The dye composition below was prepared:

| | | |
|---|---|---|
| 1-(4′-Amino-3′-methylphenyl)-4-hydroxy- | 0.837 | g |
| 2-methylpyrrolidine dihydrochloride [substantituted | | |
| para-phenylenediamine derivative of formula (I) | | |
| in accordance with the invention] | | |
| 2,4-Diamino-1-(β-hydroxyethyloxy)benzene | 0.723 | g |
| dihydrochloride | | |
| Alkyl C₈-C₁₀ polyglucoside as an aqueous 60% | 3.24 | g AM* |
| solution, sold under the name Oramix CG 110 ® by | | |
| the company SEPPIC | | |
| Ethanol | 18 | g |
| Benzyl alcohol | 1.8 | g |
| Polyethylene glycol 400 | 2.7 | g |
| Pentasodium salt of diethylenetriaminepenta- | 0.43 | g AM* |
| acetic acid as an aqueous 40% solution, sold | | |
| under the name Dissoluine D-40 ® by the company | | |
| Akzo | | |
| Sodium metabisulphite | 0.205 | g |
| Aqueous ammonia containing 20.5% NH₃.p | 10 | g |
| Demineralized water qs | 100 | g |

AM* denotes Active Material

At the time of use, the dye composition described above was mixed weight for weight with a 20-volumes hydrogen peroxide solution (6% by weight).

The mixture thus prepared was applied for 30 minutes to locks of permanent-waved natural gray hair containing 90% white hairs. The locks were then rinsed, washed with a standard shampoo, rinsed again and then dried.

The hairs were dyed a bright blue shade.

1. A ready-to-use composition for the oxidation dyeing of keratin fibers, in particular of human keratin fibers such as the hair, characterized in that it comprises, in a medium that is suitable for dyeing:
(A) at least one oxidation base chosen from the substituted para-phenylenediamine derivatives of formula (I) below, and the addition salts thereof with an acid: 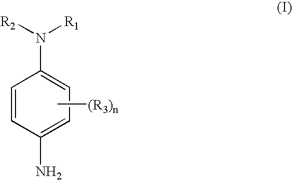
in which:
R₁ and R₂ can take one of the following meanings i) to v) below:
i) R₁ and R₂ simultaneously represent a —(CH₂)₂CHOHCH₂OH radical; or
ii) R₁ represents a —CH₂(CHOH)₄CH₂OH radical and R₂ represents a hydrogen atom, an alkyl or aryl radical or a heterocycle; or
iii) R₁ represents an alkyl or aryl radical or a heterocycle and R₂ represents an alkylene radical —(CH₂)ₘ— in which m is an integer equal to 2 or 3, said alkylene radical forming a ring together with the nitrogen atom, the carbon atom of the benzene ring bearing the nitrogen atom and one of the two carbon atoms of the benzene ring that are adjacent to it, it being understood that when R₁ is an alkyl or aryl radical, then either R₁ or said alkylene radical is substituted with a radical containing at least one nitrogen, oxygen or sulfur atom;
iv) R₁ represents a radical —(CH₂CH₂O)ₚR₄ in which p is an integer between 2 and 8 inclusive, R₄ and R₂, which may be identical or different, represent a hydrogen atom, an alkyl or aryl radical or a heterocycle;
v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;
R₃ represents a halogen atom, an alkyl or aryl radical, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical, a heterocycle or a heterocycle linked to the benzene ring of formula (I) via an ether or thio bond;
said alkyl radicals containing from 1 to 25 carbon atoms and possibly being linear, branched or cyclic and possibly being substituted with one or more radicals, and in this case possibly representing a mono- or polyhydroxyalkyl radical, an alkoxyalkyl radical, an aminoalkyl radical optionally substituted on the nitrogen atom, or a carboxyalkyl, alkylcarboxyalkyl, thioalkyl, alkylthioalkyl, cyanoalkyl, trifluoroalkyl, sulfoalkyl, phosphoalkyl or haloalkyl radical;
said alkoxy radicals containing from 1 to 25 carbon atoms and possibly being linear, branched or cyclic;
said aryl radicals containing from 6 to 26 carbon atoms and possibly being substituted with one or more radicals chosen from alkyl, substituted alkyl and alkoxy radicals;
the heterocycles being monocyclic or polycyclic, each ring being 3-, 4-, 5- or 6-membered and possibly containing one or more hetero atoms, it being understood that in the case of polycyclic heterocycles, at least one of the rings contains at least one hetero atom such as N, O or S;
n is an integer between 0 and 4; it being understood that when n is greater than 1, then the radicals R₃ may be identical or different and may together form a 3-, 4-, 5- or 6-membered saturated or unsaturated ring; with the proviso that:
1) when R₁ and R₂ have the meanings defined in point v), then the compounds of formula (I) do not contain more than 3 hydroxyl radicals;
2) when R₁ and R₂ have the meanings defined in point v) and when R₁ and R₂ form a pyrrolidine ring substituted with a carbamoyl radical on the carbon in the alpha position to the nitrogen atom to which they are attached, then n is other than 0; or the pyrrolidine ring bears at least two substituents;
3) when R₁ and R₂ have the meanings defined in point v) and when R₁ and R₂ form a pyrrolidine ring substituted with a hydroxymethyl radical on the carbon in the alpha position relative to the nitrogen atom to which they are attached and when n=0 or 1, then either said ring bears at least two additional substituents, or said ring bears only one second substituent other than a hydroxyl radical on the carbon located in the β position relative to the nitrogen atom and relative to the carbon bearing said hydroxymethyl substituent; or alternatively when R₁ and R₂ have the meanings defined in point v) and when R₁ and R₂ form a pyrrolidine ring substituted with a hydroxymethyl radical on the carbon located in the alpha position relative to the nitrogen atom to which they are attached, and when n=1, then R₃ is other than an alkyl or mono- or polyhydroxyalkyl radical;
4) when R₁ and R₂ have the meanings defined in point iii), the compounds of formula (I) must satisfy at least one of the following four conditions:
a) irrespective of the value of n, the alkylene ring formed by the radical R₂ comprises a substituent in addition to the radical R₁; or
b) n is greater than 1; or
c) when n is equal to 1, then R₃ represents an aryl radical or a heterocycle; or
d) when n is equal to 0 or 1, then R₁ represents an aryl radical, a heterocycle or a substituted alkyl radical other than a monohydroxyalkyl radical;
5) R₁ and R₂ form a ring other than piperazine or diazacycloheptane;
(B) at least one alkaline agent chosen from alkanolamines, diaminoalkanes and aqueous ammonia, and
(C) hydrogen peroxide.

2. The composition as claimed in claim 1, characterized in that, in formula (I), R₁ and R₂ take one of the meanings i) to v) below:
i) R₁ and R₂ simultaneously represent a —(CH₂)₂CHOHCH₂OH radical; or
ii) R₁ represents a —CH₂(CHOH)₄CH₂OH radical and R₂ represents a hydrogen atom or an alkyl radical; or
iv) R₁ represents a radical —(CH₂CH₂O)ₚR₄ in which p is an integer between 2 and 8 inclusive, R₄ and R₂, which may be identical or different, represent a hydrogen atom or an alkyl radical;
v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, which is not in a meta position relative to the nitrogen atom of the heterocycle;
R₃ represents a halogen atom, an alkyl or aryl radical or a heterocycle,
n is an integer equal to 0, 1 or 2.

3. The composition as claimed in claim 1 or 2, characterized in that, in formula (I), R₁ and R₂ form a pyrrolidine heterocycle.

4. The composition as claimed in claim 1 or 2, characterized in that the substituted para-phenylenediamine derivative(s) of formula (I) is (are) chosen from:
1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis-(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-β-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1-N-(2′,3′,4′,5′,6′-pentahydroxyhexyl)-1-N-(4″-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine,1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′-pentahydroxyhexyl)-3-ethyloxy-para-phenylenediamine, 1-N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)-amino-7-amino-1-methylindole, 1-N-(hydroxyethyloxyethyl)-1-N-(2′,3′,4′,5′,6′-pentahydroxyhexyl)-3-ethyl-para-phenylenediamine, 1-N-(3′,4′-dihydroxybutyl)-5-aminoindoline, 1-(2′-hydroxyethyl)-2-methyl-5-aminoindoline, 1-methyl-2-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyl-oxyethyloxyethyloxyethyl-6-isopropyl-5-aminoindoline, 2-hydroxyethyl-3-methyl-5-aminoindoline, 2-hydroxyethyloxyethyloxyethyl)-5-aminoindoline, 1-carboxymethyl-2,3,3-trimethyl-5-aminoindoline, 1-methylsulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, the dimethyl ester 6-amino-1-methyl-1,2,3,4-tetrahydrofuro-[2,3,h]-quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetrahydroquinoline, 6-amino-1-(ethoxyethoxyethoxyethoxy-3′,4′-dihydroxybutyl)-2,2,3,7-tetramethyl-1,2,3,4-tetrahydroquinoline, 6-amino-1-(hydroxyethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyl-oxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyl-oxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyloxyethyloxyethyloxyethyloxyethyloxy-ethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyloxy-ethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl)-4-(hydroxyethyloxyethyloxyethyloxy-ethyl)-2,2-dimethyl-7-isopropyl-1,2,3,4-tetrahydroquinoline, 6-amino-1-(hydroxyethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydroquinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline, 6-amino-1-bromo-2,2-dimethyl-4-mercapto-7-isopropyloxy-1,2,3,4-tetrahydroquinoline, 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic acid), 1-(4′-amino-3′-acetylaminophenyl)-3-hydroxymethylpyrrolidine, 7-amino-4-(2′-methyl)-pyrrolidinylbenzofuran, 1-(4′-aminophenyl)-2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropylphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2″′,4″′-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxyethyloxyethyloxyethyloxyethyloxyethyloxy-ethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3-4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxyethyloxyethyloxyethyl)-3-isopropyloxy-para-phenylenediamine, 1-N,N-bis(hydroxyethyloxyethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(methoxyethyloxyethyloxyethyloxyethyloxyethyloxy-ethyloxyethyl)-3-methoxy-para-phenylenediamine, 1-N,N-bis(hydroxyethyloxyethyloxyethyloxyethyloxyethyloxy-ethyloxyethyloxyethyl)-3-methyl-para-phenylenediamine, 1-N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine and 1-N,N-bis(benzyloxyethyloxyethyloxyethyl)-3-isopropyl-para-phenylenediamine, and the addition salts thereof with an acid.

5. The composition as claimed in claim 4, characterized in that they are chosen from:
1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine and 1-N-(hydroxyethyloxyethyl)-1-N-(2′,3′,4′,5′,6′-pentahydroxyhexyl)-3-ethyl-para-phenylenediamine, and the addition salts thereof with an acid.

6. The composition as claimed in claim 4, characterized in that they are chosen from:
1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 1-(4′-aminophenyl)-2-(4″-aminophenoxymethyl)piperidine, 1-(4′-aminophenyl)-2-hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxy-ethyloxyethyloxyethyl)-3-methyl-para-phenylenediamine and 1-N,N-bis(benzyloxyethyloxyethyloxyethyl)-3-isopropyl-para-phenylenediamine.

7. The composition as claimed in any one of the preceding claims, characterized in that the addition salts with an acid are chosen from the hydrochlorides, hydrobromides, sulfates, tartrates, lactates and acetates.

8. The composition as claimed in any one of the preceding claims, characterized in that the substituted para-phenylenediamine derivative(s) of formula (I) and/or the addition salt(s) thereof with an acid represent from 0.0001% to 20% by weight relative to the total weight of the composition.

9. The composition as claimed in claim 7, characterized in that the substituted para-phenylenediamine derivative(s) of formula (I) and/or the addition salt(s) thereof with an acid represent from 0.001% to 15% by weight relative to the total weight of the composition.

10. The composition as claimed in claim 9, characterized in that the substituted para-phenylenediamine derivative(s) of formula (I) and/or the addition salt(s) thereof with an acid represent from 0.01% to 10% by weight relative to the total weight of the composition.

11. The composition as claimed in claim 1, characterized in that the alkanolamine is a saturated or unsaturated, linear or branched hydrocarbon compound containing from 2 to 100 carbon atoms and comprising (i) at least one amine function and (ii) at least one hydroxyl function not borne by the amine function.

12. The composition as claimed in claim 11, characterized in that the alkanolamine is a saturated hydrocarbon compound containing from 2 to 100 carbon atoms.

13. The composition as claimed in claims 1 or 11 or 12, characterized in that the alkanolamines are chosen from monoethanolamine, diethanolamine, triethanolamine, triisopropanolamine, 2-amino-2-methyl-1-propanol, 2-amino-2-methyl-1,3-propanediol, 2-amino-2-ethyl-1,3-propanediol, 2-amino-1-butanol, tris(hydroxymethyl)aminomethane, 2-aminoethylethanolamine, 1-diethylamino-2,3-propanediol, 2-dimethylamino-2-methyl-1-propanol, dimethylethanolamine, diethylethanolamine, ethylmonoethanolamine and methylethanolamine.

14. The composition as claimed in claim 13, characterized in that the alkanolamine is monoethanolamine.

15. The composition as claimed in claim 1, characterized in that the diaminoalkanes are of formula (II): 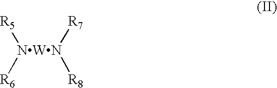
in which W is an alkylene residue optionally substituted with a hydroxyl group or a C₁-C₄ alkyl radical; R₅, R₆, R₇ and R₈, which may be identical or different, represent a hydrogen atom or a C₁-C₄ alkyl or C₁-C₄ hydroxyalkyl radical.

16. The composition as claimed in claim 15, characterized in that W is a propylene residue.

17. The composition as claimed in claim 16, characterized in that the diaminoalkane is diaminopropane.

18. The composition as claimed in claim 1, characterized in that the alkaline agent is aqueous ammonia.

19. The composition as claimed in any one of the preceding claims, characterized in that the alkaline agent(s) is (are) present in concentrations ranging from 0.1% to 20% by weight of active material relative to the total weight of the composition.

20. The composition as claimed in claim 19, characterized in that the alkaline agent(s) is (are) present in concentrations ranging from 0.5% to 10% by weight of active material relative to the total weight of the composition.

21. The composition as claimed in any one of the preceding claims, characterized in that the hydrogen peroxide is present in a concentration of between 0.5% and 15% by weight relative to the total weight of the composition.

22. The composition as claimed in claim 21, characterized in that the hydrogen peroxide is present in a concentration of between 1% and 10% by weight relative to the total weight of the composition.

23. The composition as claimed in any one of the preceding claims, characterized in that it contains at least one coupler.

24. The composition as claimed in claim 23, characterized in that the coupler(s) is (are) chosen from meta-phenylenediamines, meta-aminophenols, meta-diphenols and heterocyclic couplers, such as, for example, indole derivatives, indoline derivatives, benzimidazole derivatives, benzomorpholine derivatives, sesamol derivatives and pyridine, pyrimidine and pyrazole derivatives, and the addition salts thereof with an acid.

25. The composition as claimed in claim 24, characterized in that the coupler(s) is (are) chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one and 1-phenyl-3-methylpyrazol-5-one, and the addition salts thereof with an acid.

26. The composition as claimed in any one of claims 13 to 25, characterized in that the coupler(s) is (are) present in a concentration of between 0.0001% and 15% by weight relative to the total weight of the composition.

27. The composition as claimed in any one of the preceding claims, characterized in that it contains at least one additional oxidation base and/or at least one direct dye.

28. The composition as claimed in claim 27, characterized in that the additional oxidation base(s) is (are) chosen from para-phenylenediamine, para-tolylenediamine, 2-hydroxyethyl-para-phenylenediamine, 1-N,N-bis(2-hydroxyethyl)-para-phenylenediamine, para-aminophenols, ortho-phenylenediamines, ortho-aminophenols, double bases and heterocyclic bases.

29. The composition as claimed in claims 27 and 28, characterized in that the additional oxidation base(s) is (are) present in a concentration of between 0.0001% and 15% by weight relative to the total weight of the composition.

30. The composition as claimed in any one of the preceding claims, characterized in that the medium that is suitable for dyeing consists of water or of a mixture of water and at least one organic solvent.

31. The composition as claimed in claim 30, characterized in that the solvents are present in proportions of between 1% and 40% by weight relative to the total weight of the composition.

32. The composition as claimed in any one of the preceding claims, characterized in that it contains anionic, cationic, nonionic, amphoteric or zwitterionic surfactants or mixtures thereof, anionic, cationic, nonionic, amphoteric or zwitterionic polymers or mixtures thereof, mineral or organic thickeners, thickening polymers, antioxidants, penetrating agents, sequestering agents, fragrances, buffers, dispersants, conditioners such as, for example, volatile or non-volatile silicones, which are modified or unmodified, film-forming agents, ceramides, preserving agents and opacifiers.

33. The composition as claimed in claim 32, characterized in that it contains at least one cationic polymer in a proportion of from 0.05% to 10% by weight and at least one nonionic surfactant in a proportion of from 0.1% to 20% by weight.

34. The composition as claimed in claim 32, characterized in that it contains at least one thickening polymer comprising at least one hydrophilic unit and at least one fatty chain in a proportion of from 0.05% to 10% by weight.

35. The composition as claimed in any one of the preceding claims, characterized in that the pH is greater than 7 and preferably greater than 8.

36. The composition as claimed in any one of the preceding claims, characterized in that it is in the form of liquid, powder, cream or gel, which are optionally pressurized, or in any other form that is suitable for dyeing keratin fibers.

37. A composition intended for the oxidation dyeing of keratin fibers, and in particular of human keratin fibers such as the hair, comprising, in a medium that is suitable for dyeing, at least one substituted para-phenylenediamine derivative of formula (I) defined in any one of claims 1 to 6, and at least one alkaline agent defined in any one of claims 1 and 11 to 18.

38. A process for dyeing keratin fibers, and in particular human keratin fibers such as the hair, characterized in that a dye composition containing, in a medium that is suitable for dyeing, at least one substituted para-phenylenediamine derivative of formula (I) defined in any one of claims 1 to 6 is mixed, at the time of use, with an oxidizing composition containing, in a medium that is suitable for dyeing, at least hydrogen peroxide, the alkaline agent(s) defined in any one of claims 1 and 11 to 18 being present in one or both of the compositions, the mixture obtained is applied to the keratin fibers and is left in place for 3 to 50 minutes, after which the fibers are rinsed, washed with shampoo, rinsed again and dried.

39. A multi-compartment dyeing device or “kit” comprising at least one compartment containing a dye composition containing at least one substituted para-phenylenediamine derivative of formula (I) defined in any one of claims 1 to 6, and another compartment containing an oxidizing composition containing hydrogen peroxide, the alkaline agent(s) defined in any one of claims 1 and 11 to 18 being present in one or both of the compositions.
